(19) United States
(12) Patent Application Publication
(10) Pub. No.: US 2026/0102133 A1
(43) Pub. Date: Apr. 16, 2026

(54) DYNAMIC IMAGE PROCESSING APPARATUS, DYNAMIC IMAGE PROCESSING METHOD, AND STORAGE MEDIUM

(71) Applicant: KONICA MINOLTA, INC., Tokyo (JP)

(72) Inventors: Ryoichi WATANABE, Tokyo (JP); Kojiro ONO, Narita-shi (JP)

(73) Assignee: KONICA MINOLTA, INC., Tokyo (JP)

(21) Appl. No.: 19/353,492

(22) Filed: Oct. 8, 2025

(30) Foreign Application Priority Data
Oct. 16, 2024 (JP) 2024-180691

(51) Int. Cl. A61B 6/00; A61B 6/50; G06T 7/00; G06T 7/246

(52) U.S. Cl. CPC A61B 6/5217; A61B 6/504; G06T 7/0016; G06T 7/248; G06T 2200/28; G06T 2207/10116; G06T 2207/30061; G06T 2207/30104

(57) ABSTRACT

A dynamic image processing apparatus includes a hardware processor. The hardware processor performs the following, acquiring a dynamic image including a plurality of frame images obtained by dynamic imaging using radiation, setting a certain region of interest in each of the plurality of frame images that are acquired, and generating waveform information indicating a change in a signal value of each pixel in the set region of interest of the plurality of frame images. The hardware processor tracks the region of interest set in a standard frame image among the plurality of frame images with respect to another frame image to set the region of interest in the another frame image.

### CROSS-REFERENCE TO RELATED APPLICATION

The entire disclosure of Japanese Patent Application No. 2024-180691, filed on Oct. 16, 2024, including description, claims, drawings and abstract is incorporated herein by reference.

### BACKGROUND OF THE INVENTION

### Technical Field

The present invention relates to a dynamic image processing apparatus, a dynamic image processing method, and a storage medium.

### Description of Related Art

Clinical research on dynamic analysis using dynamic X-ray dynamic images is underway, and the dynamic analysis is being utilized in examinations of various diseases. For example, a dynamic analysis method for analyzing blood flow such as pulmonary blood flow and cardiac blood flow using the X-ray dynamic image has been developed. Japanese Patent No. 7424423 describes a dynamic image analysis apparatus that generates information on pulmonary valve regurgitation based on a dynamic image of a site related to at least one of a pulmonary artery or a heart.

In the dynamic analysis processing, a predetermined region of interest is set in each frame image of the dynamic image, and a blood flow waveform or the like is observed from a change in a signal value of each region of interest. Here, when the region of interest is set, an unpredictable movement of the patient, for example, a body movement or the like due to a cough or the like may occur. When an unpredictable movement of the patient occurs, a position of the region of interest shifts between the frame images, and a change or the like in the signal value of an unexpected region different from the region of interest set in the frame image may be observed. As a result, a false analysis occurs, and thus it is difficult to obtain an appropriate analysis result of an expected region.

### SUMMARY OF THE INVENTION

Therefore, in order to solve the above-described problem, an object of the present invention is to provide a dynamic image processing apparatus, a dynamic image processing method, and a storage medium that can acquire dynamic images appropriate for dynamic analysis processing.

A dynamic image processing apparatus according to one aspect of the present invention is the dynamic image processing apparatus including:
a hardware processor that is configured to perform,
   acquiring a dynamic image including a plurality of frame images obtained by dynamic imaging using radiation,
   setting a certain region of interest in each of the plurality of frame images that are acquired, and generating waveform information indicating a change in a signal value of each pixel in the set region of interest of the plurality of frame images,
wherein the hardware processor tracks the region of interest set in a standard frame image among the plurality of frame images with respect to another frame image to set the region of interest in the another frame image.

A dynamic image processing method according to another aspect of the present invention is a dynamic image processing method including:
acquiring a dynamic image including a plurality of frame images obtained by dynamic imaging using radiation,
setting a certain region of interest in each of the plurality of frame images that are acquired, and
generating waveform information indicating a change in a signal value of each pixel in the set region of interest of the plurality of frame images,
wherein in the setting, the region of interest set in a standard frame image among the plurality of frame images is tracked with respect to another frame image to set the region of interest in the another frame image.

A storage medium according to an aspect of the present invention is a non-transitory computer readable storage medium including a program that controls a computer to perform,
acquiring a dynamic image including a plurality of frame images obtained by dynamic imaging using radiation,
setting a certain region of interest in each of the plurality of frame images that are acquired, and
generating waveform information indicating a change in a signal value of each pixel in the set region of interest of the plurality of frame images,
wherein in the setting, the region of interest set in a standard frame image among the plurality of frame images is tracked with respect to another frame image to set the region of interest in the another frame image.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 is a diagram illustrating an example of a schematic configuration of an image capturing system according to a first embodiment;

FIG. 2 is a flowchart illustrating an example of an operation of a dynamic analysis apparatus in performing tracking of a region of interest set in a frame image serving as a standard on another frame image according to the first embodiment;

FIG. 3 is a diagram illustrating an example of a standard frame image in which a region of interest is set according to the first embodiment;

FIG. 4 is a diagram illustrating an example of a second frame image in which a region of interest is set by tracking of a template image set in the standard frame image according to the first embodiment;

FIG. 5 is a diagram illustrating an example of a third frame image in which the region of interest is set by tracking of the template image set in the standard frame image according to the first embodiment;

FIG. 6 is a signal value change waveform showing a change in a signal value of a pixel in the region of interest of a dynamic image according to the first embodiment;

FIG. 7 is a flowchart illustrating an example of the operation of the dynamic analysis apparatus when tracking of the region of interest set in the standard frame image is performed on another frame image according to the second embodiment;

FIG. 8 is a flowchart illustrating an example of the operation of the controller during first processing according to the second embodiment;

FIG. 9 is a diagram illustrating the signal value change waveform in which the standard frame waveform and the like are set according to the second embodiment;

FIG. 10 is a flowchart illustrating an example of the operation of the controller during second processing according to the second embodiment; and

FIG. 11 is a diagram illustrating the signal value change waveform in which a first frame waveform, a second frame waveform, a third frame waveform, and a fourth frame waveform are set in each respiratory cycle according to the second embodiment.

### DETAILED DESCRIPTION

Hereinafter, one or more embodiments of the present disclosure will be described with reference to the drawings. However, the scope of the invention is not limited to the disclosed embodiments.

Below, with reference to the accompanying drawings, a dynamic image processing apparatus, a dynamic image processing method, and a storage medium according to preferred embodiments of the present disclosure are described in detail.

### FIRST EMBODIMENT

### Example of Configuration of Image Capturing System 100

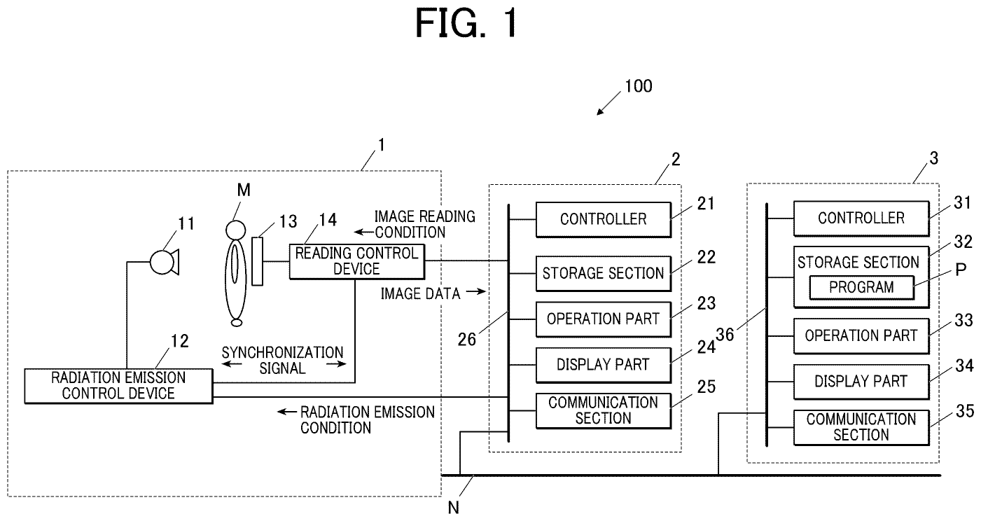

FIG. 1 is a diagram illustrating an example of a schematic configuration of an image capturing system 100 according to a first embodiment. The image capturing system 100 includes an imaging apparatus 1, a console 2, and a dynamic analysis apparatus 3 that is an example of a dynamic image processing apparatus. The imaging apparatus 1, the console 2, and the dynamic analysis apparatus 3 are communicably connected to each other via a network N such as a Local Area Network (LAN). A communication method of the network N may be wired communication or wireless communication.

The imaging apparatus 1 captures a dynamic image of a predetermined imaging area of a subject M. The console 2 controls radiographic imaging by the imaging apparatus 1 and controls a reading operation of a radiographic image by the imaging apparatus 1. The dynamic analysis apparatus 3 performs predetermined dynamic analysis processing on the dynamic image transmitted from the console 2 or the like. According to the present embodiment, the dynamic analysis apparatus 3 executes preprocessing for reducing noise due to body movement fluctuation or the like of the subject M in the region of interest of the dynamic image before performing the dynamic analysis processing. The region of interest is referred to as an ROI (Region of Interest). The apparatuses included in the image capturing system 100 comply with the DICOM standard, and communication between the apparatuses is performed in accordance with the DICOM standard. DICOM is an abbreviation for Digital Image and Communications in Medicine.

### Example of Configuration of Imaging Apparatus 1

The imaging apparatus 1 can perform dynamic imaging of, for example, morphological changes in expansion and contraction of lungs due to respiratory motion and beating of a heart. The dynamic imaging refers to acquiring a series of images of the subject M by repeatedly irradiating the subject M with pulsed radiation such as X-rays at predetermined time intervals in response to one imaging operation. Repeatedly irradiating pulsed radiation at predetermined time intervals is referred to as pulsed irradiation. Alternatively, the dynamic imaging refers to acquiring a series of images of the subject M by continuously irradiating the subject M with a low dose rate without interruption in response to one imaging operation. Continuously applying radiation without interruption is referred to as continuous irradiation. The series of images obtained by dynamic imaging is called a dynamic image. Each of all of the images constituting the dynamic image is called a frame image. Here, the dynamic imaging includes moving image capturing, but does not include capturing a still image while displaying a moving image. Further, examples of a dynamic image include a moving image but do not include images obtained by capturing still images while displaying the moving image.

As shown in FIG. 1, the imaging apparatus 1 includes a radiation source 11, a radiation emission control device 12, a radiation detector 13, and a reading control device 14. The radiation emission control device 12 and the reading control device 14 are connected to each other via a communication cable or the like, and exchange synchronization signals with each other, thereby synchronizing a radiation emission operation and an image reading operation. The radiation detector 13 and the reading control device 14 may be integrally configured.

The radiation source 11 is arranged at a position facing the radiation detector 13 with the subject M interposed therebetween. The radiation source 11 irradiates the subject M with radiation such as X-rays under the control of the radiation emission control device 12. The radiation emission control device 12 is connected to the console 2. The radiation emission control device 12 controls the radiation source 11 on the basis of a radiation emission condition input from the console 2 to perform radiographic imaging. The radiation emission conditions input from the console 2 include, for example, a pulse rate, a pulse width, a pulse interval, the number of imaging frames per imaging, a value of an X-ray tube current, a value of an X-ray tube voltage, and a type of an additional filter. The pulse rate is the number of times that radiation is emitted per second, and matches a frame rate described below. The pulse width is a radiation irradiation time per radiation irradiation. The pulse interval is an amount of time from start of one radiation irradiation to start of the next radiation irradiation, and matches a frame interval described below.

The radiation detector 13 may include a semiconductor image sensor such as a flat panel detector (FPD). The FPD includes a substrate and the like formed of glass or the like. At predetermined positions on the substrate, a plurality of detection elements including pixels and the like are arranged in a matrix. The plurality of detection elements detect radiation emitted from the radiation source 11 and transmitted through at least the subject M in accordance with the intensity of the radiation, convert the detected radiation into an electrical signal, and accumulate the electrical signal. Each pixel includes a switching section such as a thin film transistor (TFT). Examples of a type of FPD include an indirect conversion type and a direct conversion type, and any type may be used. The indirect conversion type is a method in which the radiation is converted into the electrical signal by a photoelectric conversion element via a scintillator. The direct conversion type is a method of directly converting the radiation into the electrical signal.

The reading control device 14 is connected to the console 2. The reading control device 14 controls the switching section of each pixel of the radiation detector 13 based on an image reading condition input from the console 2. The reading control device 14 switches reading of the electrical signal accumulated in each pixel of the radiation detector 13, and acquires image data by reading the electrical signal accumulated in the radiation detector 13. The image data is each frame image of the dynamic image or a still image. If a structure exists between the radiation source 11 and the radiation detector 13, the amount of radiation reaching the radiation detector 13 decreases due to the structure. In this case, the signal value of each pixel of the image data changes according to the structure of the subject M. The signal value includes a pixel value, a density value, and the like. The reading control device 14 outputs the acquired dynamic image or still image to the console 2. The image reading condition includes, for example, a frame rate, a frame interval, a pixel size, and an image size. The frame rate is the number of frames acquired per second, and coincides with the pulse rate. The frame interval is the amount of time from the start of the operation of acquiring one frame image to the start of the operation of acquiring the next frame image, and matches with the pulse interval.

### Configuration Example of Console 2

The console 2 is, for example, a computer such as a personal computer, a workstation, or the like. As illustrated in FIG. 1, the console 2 includes a controller 21, a storage section 22, an operation part 23, a display part 24, and a communication section 25. The controller 21, the storage section 22, the operation part 23, the display part 24, and the communication section 25 are connected by wiring such as a bus 26.

The controller 21 includes a CPU (Central Processing Unit), a RAM (Random Access Memory), and the like. The CPU 21 reads a system program and various processing programs stored in the storage section 22 in response to an operation of the operation part 23, develops the programs in the RAM, and executes various processes in accordance with the developed programs. The controller 21 centrally controls operation of sections of the console 2 and the radiation emission operation and the reading operation of the imaging apparatus 1.

The storage section 22 is a nonvolatile semiconductor memory, a hard disk, or the like. The storage section 22 stores various programs to be executed by the controller 21, parameters required for execution of processing by the programs, and data such as processing results. The various programs are stored in the form of readable program codes. The controller 21 sequentially performs operations in accordance with the program codes.

The operation part 23 includes a keyboard, a mouse, and the like. The operation part 23 may be a touch screen combined with a display screen of the display part 24. The operation part 23 accepts various instructions by a user's input operation, and outputs an instruction signal corresponding to the accepted instruction to the controller 21.

The display part 24 is a monitor such as a liquid crystal display (LCD). The display part 24 displays an input instruction from the operation part 23, data, and the like in accordance with an instruction of a display signal input from the controller 21.

The communication section 25 includes a LAN adapter and a modem. The communication section 25 transmits and receives signals, data, and the like to and from the imaging apparatus 1, the dynamic analysis apparatus 3, and the like connected to the network N.

### Configuration Example of Dynamic Analysis Apparatus 3

The dynamic analysis apparatus 3 is used as a diagnosis support apparatus for supporting diagnosis by a doctor. The dynamic analysis apparatus 3 is constituted by, for example, a computer such as a personal computer or a workstation. As illustrated in FIG. 1, the dynamic analysis apparatus 3 includes a controller 31 (hardware processor), a storage section 32, an operation part 33, a display part 34, and a communication section 35. The controller 31, the storage section 32, the operation part 33, the display part 34, and the communication section 35 are connected by wiring of a bus 36.

The controller 31 includes a CPU, a RAM, and the like. The CPU reads various programs P such as a system program stored in the storage section 32 in response to an operation of the operation part 33, develops the programs in the RAM, and executes various processes in accordance with the developed programs. The controller 31 centrally controls the operation of each part of the dynamic analysis apparatus 3.

The storage section 32 includes a nonvolatile semiconductor memory, a hard disk, and the like. The storage section 32 stores various programs P to be executed by the controller 31, parameters required for execution of processing by the programs P, and data such as processing results. The various programs P are stored in the form of readable program codes. The controller 31 sequentially executes operations according to the program code.

The operation part 33 includes the keyboard, the mouse, and the like. The operation part 33 may be the touch screen combined with the display screen of the display part 24. The operation part 33 accepts various instructions by the user's input operation, and outputs the instruction signal corresponding to the accepted instruction to the controller 31.

The display part 34 includes the monitor such as an LCD. The display part 34 performs various displays in accordance with an instruction of a display signal input from the controller 31. The communication section 35 includes the LAN adapter and the modem. The communication section 35 transmits and receives signals, data, and the like to and from the console 2 and the like connected to the network N.

The controller 31 of the dynamic analysis apparatus 3 functions as at least an acquisition section, a setting section, a generating section, and an extraction section. The controller 31 including a processor that realizes functions of the acquisition section, the setting section, the generation section, the extraction section, and the like by executing a program P stored in the storage section 32 or the like. The acquisition section acquires the dynamic image composed of a plurality of frame images obtained by the dynamic imaging using the radiation. The setting section sets a predetermined region of interest in each of the plurality of frame images acquired by the acquisition section. Specifically, the setting section sets the region of interest, which is a target observation site, in one standard frame image among the plurality of frame images. The region of interest is a local region in the frame image. Hereinafter, the frame image serving as a standard is referred to as a standard frame image.

Next, the setting section tracks the region of interest set in the standard frame image with respect to another frame image, and sets the region of interest in another frame image. This is because, at the time of dynamic imaging, random body movement fluctuation occurs due to unpredictable movement by the patient, coughing, poor breath holding, or the like, and the region of interest may move between frame images. The tracking of the region of interest with respect to another frame image is processing for achieving the dynamic analysis processing with high accuracy, and is preprocessing to be executed before the dynamic analysis processing. The generating section generates a signal value change waveform (dose waveform) as waveform information indicating a change in the signal value of each pixel in the region of interest of the plurality of frame images set by the setting section. The extraction section extracts the frame image including a dose fluctuation different from the blood flow fluctuation and the respiration-induced fluctuation included in the signal value change waveform generated by the generating section. For example, the extraction section extracts noise due to periodic or random body motion that could not be identified by tracking by the setting section.

### Operation Example of Image Capturing System 100

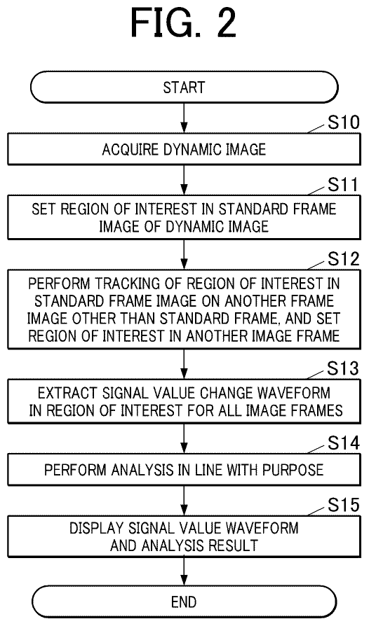

FIG. 2 is a flowchart illustrating an example of an operation of a dynamic analysis apparatus 3 in performing tracking of a region of interest set in a frame image serving as a standard on another frame image according to the first embodiment. The controller 31 implements each process including an acquisition step, a setting step, a generation step, and an extraction step by executing the program P stored in the storage section 32. Hereinafter, the frame image serving as the standard is referred to as the standard frame image.

First, the operation of the console 2 during dynamic imaging will be described. The controller 21 of the console 2 acquires the image data of a plurality of frame images captured by the imaging apparatus 1. The controller 21 stores each acquired frame image in the storage section 22 in association with a frame number indicating the imaging order. Next, the controller 21 displays a dynamic image including the acquired frame image on the screen of the display part 24. The user checks whether the image is suitable for diagnosis. The controller 21 adds patient information, inspection information, and the like to the dynamic image on the basis of a confirmation instruction by the user, and transmits the dynamic image to which the patient information and the like are added to the dynamic analysis apparatus 3.

As illustrated in FIG. 2, the controller 31 of the dynamic analysis apparatus 3 acquires the dynamic image of a predetermined imaging area from the console 2 via the communication section 35 (step S10). The imaging area is, for example, a front of a chest as described later. The dynamic image is composed of a plurality of frame images. The controller 31 stores the acquired dynamic image in the storage section 32.

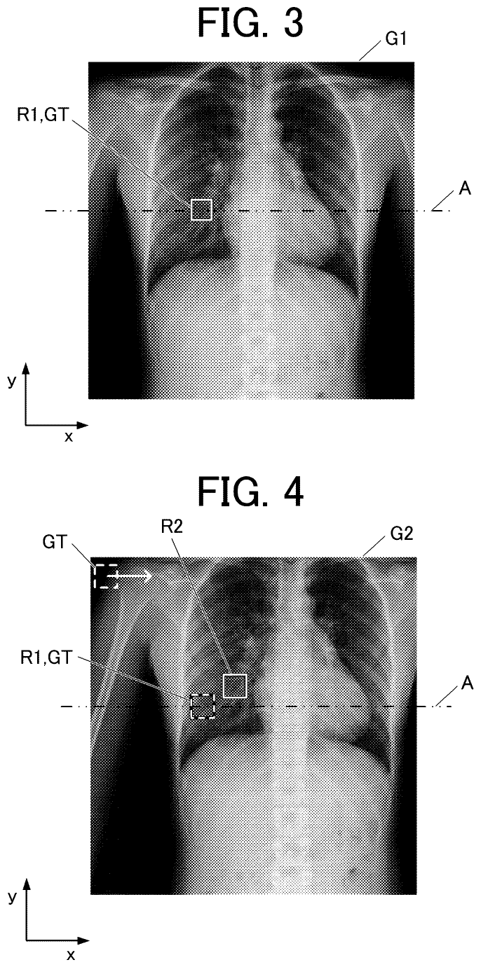

The controller 31 sets a predetermined region of interest in the standard frame image constituting the acquired dynamic image (step S11). FIG. 3 is a diagram showing an example of a standard frame image G1 in which a region of interest R1 is set according to the first embodiment. Note that in FIG. 3, a horizontal direction of the standard frame image G1 is defined as an X direction, and a vertical direction is defined as a Y direction. The controller 31 sets the first frame image among the acquired plurality of frame images as the standard frame image G1. The controller 31 sets the region of interest R1 in the standard frame image G1. For example, the controller 31 sets the region of interest R1 in a vicinity of the pulmonary artery in a lung field which is a target observation site. The vicinity of the pulmonary artery is a portion with a large blood flow and appears whitish in the dynamic image. Therefore, the controller 31 can automatically set, as the region of interest R1, the region in which many pixels having high luminance values are gathered in the lung field region in the dynamic image. Note that the setting of the region of interest R1 is not limited to the automatic setting by the controller 31. The user such as the radiologist may manually set the region of interest R1. In this case, for example, the display part 34 may be caused to display the image of the lung field region in which a contrast between a portion that looks white due to the blood flow and the other portion is illustrated in an easily understandable manner. The user can set the region of interest R1 by selecting the vicinity of the pulmonary artery in the image displayed on the display part 34 with the operation part 33. Subsequently, the controller 31 cuts out the image in the set region of interest R1 and sets the cut-out image as a template image GT. According to the present embodiment, the template image GT has a size equal to or substantially equal to that of the region of interest R1.

Further, the controller 31 may set a plurality of regions of interest R in the standard frame image G1. For example, when the number of regions of interest R set in the standard frame image G1 is one, the motion may be respiration rather than the body motion depending on the location where the region of interest R is set. In this case, the tracking of the region of interest R between the frame images is not necessarily performed in accordance with the body motion. Therefore, the controller 31 may set a plurality of regions of interest R in the standard frame image G1, and determine that the tracking between the frame images is based on the body motion when the same motion is observed in many of the regions of interest R between the frame images. By setting a plurality of regions of interest R in this way, the body motion can be determined more accurately in some cases.

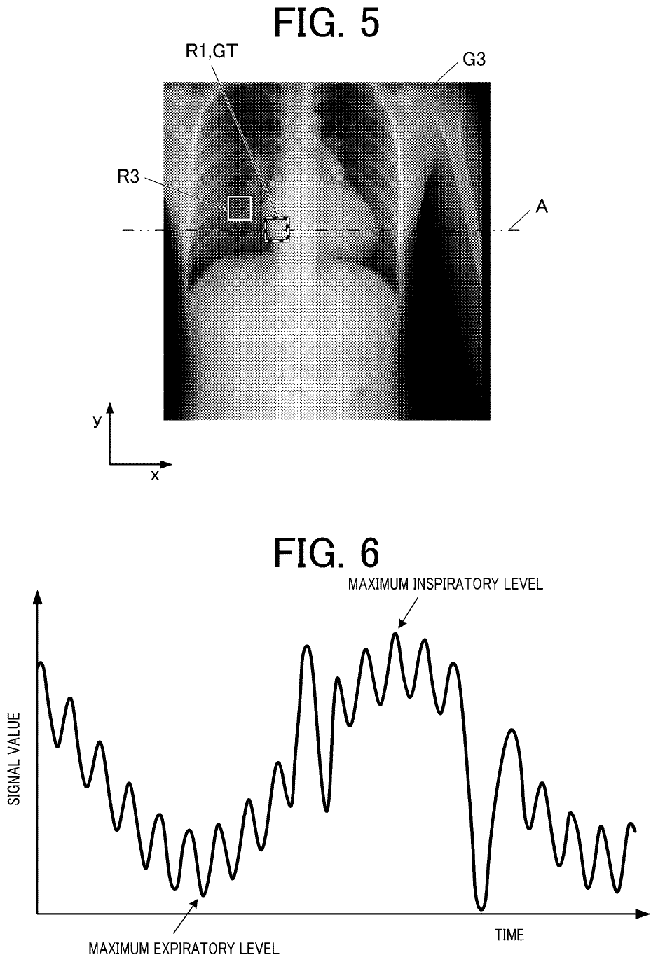

The controller 31 performs tracking of the region of interest R1 in the standard frame image G1 on another frame image other than the standard frame image in the dynamic image, and sets the region of interest in another frame image (step S12). As a method of specifying the region of interest from each frame image, for example, template matching can be used. FIG. 4 is a diagram illustrating an example of a second frame image G2 in which a region of interest R2 is set by tracking of the template image GT set in the standard frame image G1. FIG. 5 is a diagram illustrating an example of a third frame image G3 in which a region of interest R3 is set by tracking of the template image GT set in the standard frame image G1. Note that in FIG. 4 and FIG. 5, the horizontal direction of the standard frame image G1 is defined as the X direction, and the vertical direction is defined as the Y direction.

First, the controller 31 uses the template image GT set in the standard frame image G1 to set a region of interest R2 in the second frame image G2. As shown in FIG. 4, in the frame image G2, the controller 31 shifts the template image GT in the x direction one pixel at a time from the upper left end of the frame image G2. When the scanning of the template image GT reaches the right end of the frame image G2, the controller 31 shifts the template image GT by one pixel in the Y direction and then by one pixel at a time in the X direction. In this way, the controller 31 collates the template image GT with the entire frame image G2, thereby specifying a position having the highest similarity to the template image GT in the frame image G2. The controller 31 sets the identified position as the region of interest R2 in the frame image G2.

The position having the highest similarity to the template image GT in the frame image G2 can be calculated by a following equation (1). In the equation (1) below, a smaller value of Sum of Squared Difference (SSD) indicates a higher degree of similarity between the two images.

$$[ Equation 1 ]$$

$${R}_{SSD} ( x , y ) = {\sum }_{\begin{matrix}j & 0\end{matrix}}^{\begin{matrix}h & 1\end{matrix}} {\sum }_{\begin{matrix}i & 0\end{matrix}}^{\begin{matrix}w & 1\end{matrix}} {( I ( x + i , y + j ) − T ( i , j ) )}^{2} \qquad ( 1 )$$

Further, the position having the highest similarity with the template image GT in the frame image G2 may be calculated by the following equation (2). Also in the following equation (2), the smaller value of Sum of Absolute Difference (SAD), indicates the higher degree of similarity between the two images. In equation (2), since a squared error is not used, a difference between some abnormality pixel values can be allowed.

$$[ Equation 2 ]$$

$${R}_{S A D} ( x , y ) = {\sum }_{\begin{matrix}j & 0\end{matrix}}^{\begin{matrix}h & 1\end{matrix}} {\sum }_{\begin{matrix}i & 0\end{matrix}}^{\begin{matrix}w & 1\end{matrix}} \vert ( I ( x + i , y + j ) − T ( i , j ) ) \vert \qquad ( 2 )$$

As shown in FIG. 4, the controller 31 specifies a position on the diagonal upper right side of the position of the region of interest R1 set in the standard frame image G1 as a position having the highest similarity to the template image GT. It can be said that the region of interest R1 of the standard frame image G1 has moved in a diagonally right direction with respect to a standard line A of the first standard frame image G1 from the standard frame image G1 to the frame image G2 due to the body motion of the patient. Therefore, the controller 31 sets a position diagonally above and to the right of the region of interest R1 in the standard frame image G1 as the region of interest R2 in the frame image G2.

Subsequently, the controller 31 specifies a region of interest R3 in the third frame image G3. As described above, the controller 31 collates the template image GT with the entire frame image G3. As illustrated in FIG. 5, the controller 31 identifies a position diagonally above and to the left of the position of the region of interest R1 set in the standard frame image G1 as a position having the highest similarity to the template image GT. The position having the highest similarity to the template image GT can be specified by the equation (1) or equation (2) described above. It can be said that the region of interest R1 of the standard frame image G1 moves obliquely upward to the left with respect to the standard line A of the standard frame image G1 from the standard frame image G1 to the frame image G3 due to the body motion of the patient. Therefore, the controller 31 sets a position on the diagonally upper left side of the region of interest R1 in the standard frame image G1 as the region of interest R3 in the frame image G3.

As a method of identifying the region of interest from each frame image, other than template matching, for example, a technique of extracting a feature point in the frame image can be used. To be specific, the controller 31 extracts the feature point from the first standard frame image G1 constituting the dynamic image, and sets the extracted feature point as the region of interest R1. Similarly, the controller 31 extracts the feature point from another frame image G2 and the like constituting the dynamic image. The controller 31 matches the feature point of the standard frame image G1 with the feature point extracted from another frame image, extracts the feature point having high similarity from another frame image, and sets the extracted feature point as the region of interest. Thus, the controller 31 can track the region of interest between the frame images.

The controller 31 generates a signal value change waveform indicating a variation in the signal value of each pixel in the region of interest for all the image frames (step S13). FIG. 6 is the signal value change waveform showing the change in the signal value of the pixel in the region of interest R of the dynamic image according to the first embodiment. In FIG. 6, the vertical axis represents the signal value of the pixel, and the horizontal axis represents time. The signal value change waveform is, for example, a graph obtained by averaging the signal values of the pixels in the region of interest R. The signal value change waveform includes at least the respiration-induced fluctuation associated with inhalation and exhalation of the subject M and the blood flow fluctuation associated with pulsation of the heart (heartbeat).

The respiration-induced fluctuation will be described. Air flows into a lung field during inhalation time from maximum exhalation to maximum inhalation. A maximum expiratory level is a timing at which the air in the lung field is fully exhaled. A maximum inspiratory level is the timing at which the air in the lung field is fully inhaled. In this case, the amount of transmitted X-rays is large in the lung field region, and the signal value of the pixel in the region of interest R increases from the maximum expiratory level toward the maximum inspiratory level. On the other hand, the air in the lung field flows out during an exhalation period from the maximum inhalation to the maximum exhalation. In this case, the amount of transmitted X-rays is small in the lung field region, and the signal value of the pixel in the region of interest R in the dynamic image decreases from the maximum inhalation toward the maximum exhalation. Therefore, as illustrated in FIG. 6, the respiration-induced fluctuation has a waveform that gradually changes between the maximum expiratory level and the maximum inspiratory level in accordance with inhalation and exhalation of the patient.

Subsequently, the blood flow fluctuation will be described. When the heart is in ventricular diastole, less blood flows into the lung field. In this case, the amount of transmitted X-rays increases in the pulmonary artery, and the signal value of each pixel in the region of interest R of the dynamic image also increases. On the other hand, when the heart is in ventricular systole, a large amount of blood flows into the lung field from the heart via the pulmonary artery. Therefore, the amount of transmitted X-rays decreases in the lung field region, and the signal value of each pixel in the region of interest R of the dynamic image also decreases. Therefore, as shown in FIG. 6, the blood flow fluctuation has a waveform in which the signal value repeatedly increases and decreases in accordance with the heartbeat of the heart, and is superimposed on the waveform of the respiration-induced fluctuation.

The controller 31 executes the dynamic analysis processing in line with the purpose using the generated signal value change waveform (step S14). For example, when the region of interest R is the pulmonary artery, the controller 31 calculates and analyzes a signal value change amount, a signal value change rate, or the like of the pulmonary artery to calculate a feature serving as an index of a pulmonary circulation blood volume or the like. When the region of interest R is the heart, the controller 31 calculates and analyzes a signal change amount, the signal value change rate, or the like of the heart to calculate the feature amount serving as the index of the cardiac function or the like.

The controller 31 causes the generated signal value change waveform and the dynamic analysis result to be displayed on the screen of the display part 34 (step S15). Note that the signal value change waveform and the dynamic analysis result may be displayed on a part other than the display part 34 of the dynamic analysis apparatus 3. For example, the signal value change waveform and the like may be displayed on a display device such as the display part 24 of the console 2.

According to the first embodiment, the tracking of the region of interest R1 in the standard frame image G1 is performed on another frame image G2 or the like, so that the region of interest R2 or the like is set in another frame image G2 or the like. Thus, even when the region of interest R2 or the like moves due to the body motion such as coughing of the patient in another frame image G2 or the like, the movement of the patient can be followed so that the region of interest R2 or the like can be set at the expected target observation site. That is, it is possible to prevent a mismatch of the target observation site in each frame image. This makes it possible to reduce noise due to the patient's body movement or the like in each frame image of the dynamic image at a timing before the dynamic analysis processing. As a result, since a predetermined dynamic analysis processing can be performed using the appropriate dynamic image, an accurate result of the dynamic analysis processing can be obtained.

### SECOND EMBODIMENT

In the second embodiment, in addition to the tracking of the region of interest in each frame image described in the first embodiment, a frame waveform and the like containing random and periodic noise is extracted using the signal value change waveform based on the region of interest. Hereinafter, the differences from the first embodiment will be mainly described, the constituent elements substantially common to the first embodiment will be assigned with the same reference numerals, and the common description will be omitted.

### Operation Example of Image Capturing System 100

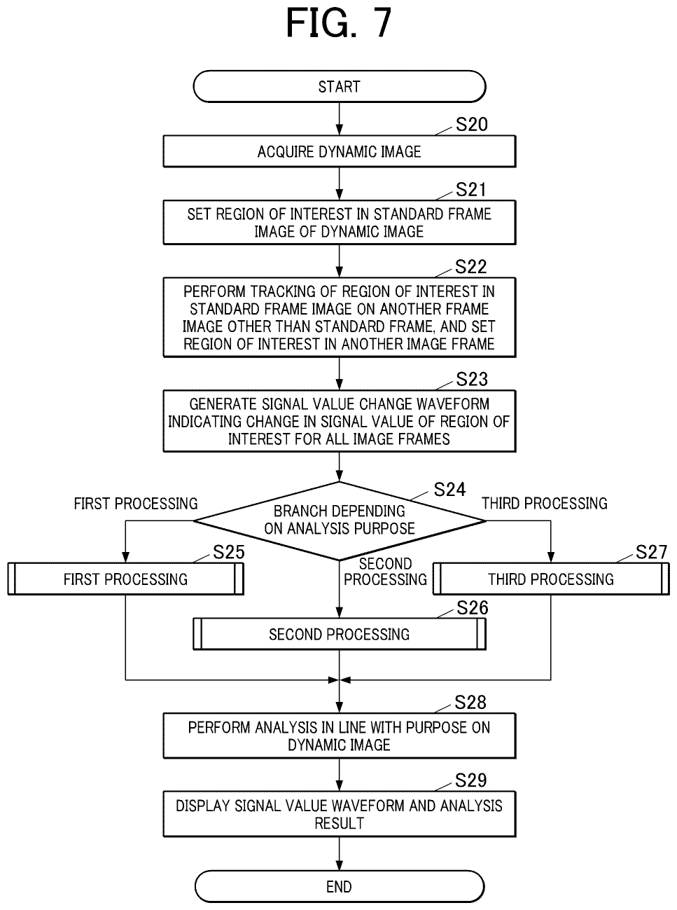

FIG. 7 is a flowchart illustrating an example of the operation of the dynamic analysis apparatus 3 when tracking of the region of interest set in the standard frame image is performed on another frame image according to the second embodiment. The controller 31 implements each process including an acquisition step, a setting step, a generation step, and an extraction step by executing the program P stored in the storage section 32.

As illustrated in FIG. 7, the controller 31 of the dynamic analysis apparatus 3 acquires the dynamic image of the predetermined imaging area from the console 2 via the communication section 35 (step S20). Next, the controller 31 sets the region of interest in the standard frame image constituting the acquired dynamic image (step S21).

The controller 31 performs tracking of the region of interest R1 in the standard frame image on another frame image other than the standard frame image in the dynamic image, and sets the region of interest in another frame image (step S22). Next, the controller 31 generates the signal value change waveform indicating the change in the signal value of each pixel in the region of interest for all the image frames (step S23).

One of the first process, the second process, and the third process is performed on the generated signal value change waveform according to the analysis purpose before the dynamic analysis processing (step S24). For example, the user may select the process suitable for the analysis purpose of the dynamic image from the items of the first to third processes displayed on the screen of the display part 34 by operating the operation part 33. In addition, the controller 31 may automatically acquire processing suitable for the analysis purpose of the acquired dynamic image on the basis of information such as the imaging area and the region of interest. Here, the first processing is processing for extracting the frame waveform including a fluctuation corresponding to the random body motion from the signal value change waveform. The second processing is processing for extracting a period having a small influence on periodic noise from the signal value change waveform. The third process is a process for extracting the frame including the body motion based on the standard frame in a case where the region of interest R is the entire image.

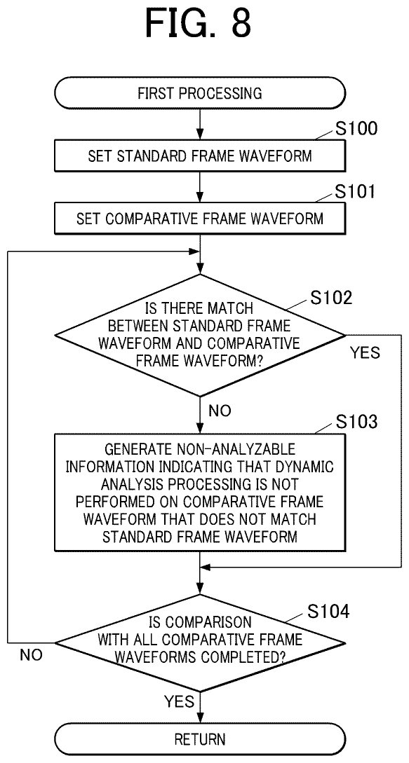

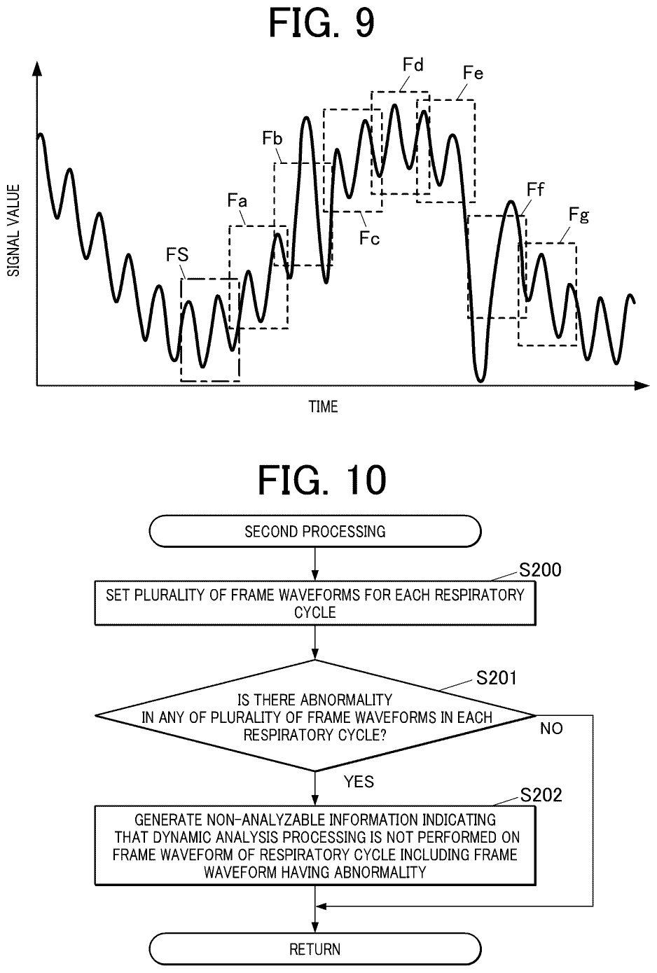

When the process branches to the first process in step S24, the controller 31 performs the first process on the acquired dynamic image (step S25). In this case, the controller 31 proceeds to the subroutine of FIG. 8. FIG. 8 is a flowchart illustrating an example of the operation of the controller 31 during first processing according to the second embodiment. As illustrated in FIG. 8, the controller 31 sets the standard frame waveform in the signal value change waveform of the region of interest in each frame image of the dynamic image (step S100). FIG. 9 is a diagram illustrating the signal value change waveform in which the standard frame waveform FS and the like are set according to the second embodiment. The standard frame waveform FS may be constituted by, for example, a plurality of frame images of the maximum expiratory level and the vicinity thereof, or may be constituted by a plurality of frame images of the maximum inspiratory level and the vicinity thereof. This is because the frame images at the maximum expiratory level and the maximum inspiratory level are least affected by the respiration-induced fluctuation, and when used in the dynamic analysis processing, an appropriate result of the dynamic analysis processing can be obtained. In the second embodiment, the standard frame waveform FS is set using a plurality of frame images at the maximum expiratory level and in the vicinity thereof. The standard frame waveform FS is set so as to include, for example, peaks corresponding to two heartbeats due to the blood flow fluctuation. In FIG. 9, a range including the standard frame waveform FS is indicated by a rectangular frame of a one dot chain line. Note that the controller 31 may set the standard frame waveform FS in accordance with the type of dynamic analysis to be performed.

The controller 31 sets the comparative frame waveform to be compared with the standard frame waveform FS in the signal value change waveform (step S101). The controller 31 may sequentially set a comparative frame waveform Fa and the like by, for example, moving the rectangular frame of the standard frame waveform FS along a time direction of the signal value change waveform. Specifically, when the standard frame waveform FS is in the 50 th to 60 th frames, the comparative frame waveforms Fa and the like can be sequentially set by moving the standard frame waveform FS every five frames in one example. In this case, the comparative frame waveform Fa is the 55 th to 65 th frames. Note that the number of frames to be moved is not limited to five frames and may be, for example, one frame. In FIG. 9, a range including the comparative frame waveforms Fa and Fb is indicated by a rectangular frame of a broken line. Note that the number of comparative frame waveforms set is not limited to the number illustrated in FIG. 9. Alternatively, the user may manually set the standard frame waveform FS, the comparative frame waveform Fa, and the like while checking the screen of the display part 34.

The controller 31 sequentially determines whether the standard frame waveform FS and the comparative frame waveform Fa or the like match each other (step S102). Specifically, the controller 31 compares the standard frame waveform FS with the comparative frame waveform Fa or the like, and determines the similarity between these frame waveforms. The controller 31 may determine the similarity to the comparative frame waveform Fa or the like using, for example, the number of peaks, the signal value, or the like of the standard frame waveform FS. For example, when determining the similarity using signal values, the controller 31 can determine that the similarity between the standard frame waveform FS and the comparative frame waveform is high when the amplitude of the comparative frame waveform is in a range of 90% to 110% with respect to the amplitude of the standard frame waveform FS being 100%. Further, the determination condition of the similarity may be a condition other than the number of peaks of the standard frame waveform FS and the width of the signal value, and may be, for example, a cross-correlation function using the entire signal value waveform.

Specifically, when the comparison target with the standard frame waveform FS is the comparative frame waveform Fa, the similarity is determined as follows. As illustrated in FIG. 9, the number of peaks of the standard frame waveform FS and the number of peaks of the comparative frame waveform Fa are two and coincide with each other within the rectangular frame. Therefore, the controller 31 can determine that the similarity between the standard frame waveform FS and the comparative frame waveform Fa is high. In this case, the controller 31 determines that the standard frame waveform FS and the comparative frame waveform Fa match each other, and proceeds to step S104.

When the comparison target with the standard frame waveform FS is the comparative frame waveform Fb, the similarity is determined as follows. As illustrated in FIG. 9, the number of peaks of the standard frame waveform FS is two within the rectangular frame, and the number of peaks of the comparative frame waveform Fb is unknown within the rectangular frame. Therefore, the controller 31 can determine that the similarity between the standard frame waveform FS and the comparative frame waveform Fb is low. The controller 31 extracts the comparative frame waveform Fb as the frame waveform including the body motion or the like of the patient. In this case, the controller 31 determines that the standard frame waveform FS and the comparative frame waveform Fa do not match, and proceeds to step S103.

The controller 31 generates non-analyzable information indicating that the comparative frame waveform Fb or the like that does not match the standard frame waveform FS is inappropriate for the dynamic analysis processing (step S103). Specifically, processing for a case where the dynamic image includes 100 frame images and 80 th to 90 th frame images include the noise due to the body movement fluctuation is executed as follows. In this case, the controller 31 generates the non-analyzable information for the 80 th to 90 th frame images and adds the generated non-analyzable information to each of the 80 th to 90 th frame images. After generating the non-analyzable information, the controller 31 proceeds to step S104.

The controller 31 determines whether the comparison of all the set comparative frame waveforms Fa and the like has been completed (step S104). When determining that the comparison of all the set comparative frame waveforms Fa and the like has not been completed, the controller 31 returns to step S102. The controller 31 moves the comparison target with the standard frame waveform FS to the adjacent comparative frame waveform or the like, and repeatedly executes the above-described comparison processing of the standard frame waveform FS. On the other hand, when determining that the comparison with all the set comparative frame waveforms Fa and the like has been completed, the controller 31 proceeds to step S28 illustrated in FIG. 7.

When the process branches to the second process in step S24, the controller 31 performs the second process on the acquired dynamic image (step S26). In this case, the controller 31 proceeds to the subroutine of FIG. 10. FIG. 10 is a flowchart illustrating an example of the operation of the controller 31 during the second processing according to the second embodiment.

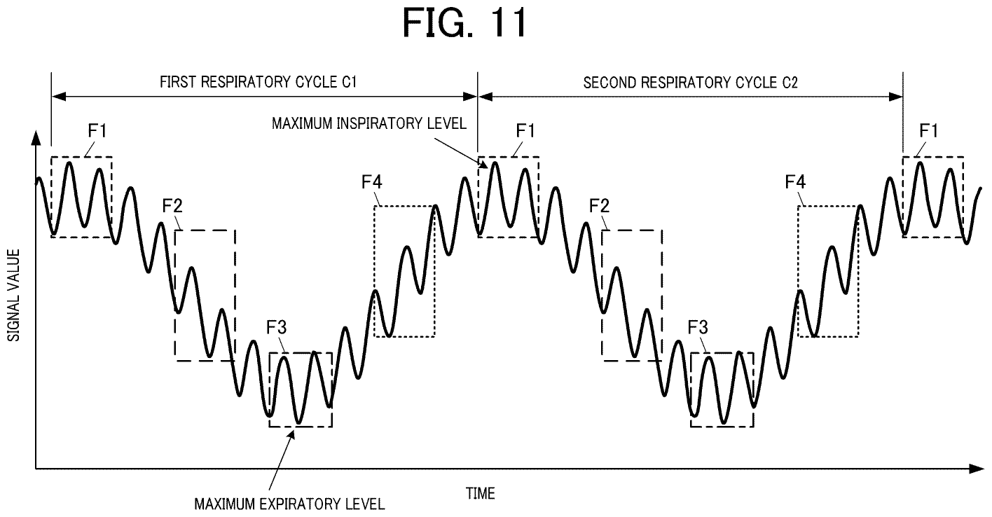

The controller 31 sets a plurality of frame waveforms in each respiratory cycle in the signal value change waveform (step S200). FIG. 11 is a diagram illustrating the signal value change waveform in which a first frame waveform F1, a second frame waveform F2, a third frame waveform F3, and a fourth frame waveform F4 are set in each respiratory cycle according to the second embodiment. In the first respiratory cycle C1, the controller 31 sets the first frame waveform F1 in the maximum inspiration period, sets the second frame waveform F2 in the expiration period, sets the third frame waveform F3 in the maximum expiration period, and sets the fourth frame waveform F4 in the inspiration period. The controller 31 sets the first frame waveform F1, the second frame waveform F2, the third frame waveform F3, and the fourth frame waveform F4 in the same manner as the first respiratory cycle C1 in the second respiratory cycle C2 and the subsequent respiratory cycles.

Here, the first frame waveform F1 is constituted by a plurality of frame images in the maximum inspiration period. The second frame waveform F2 is constituted by a plurality of frame images at a substantially intermediate position between the maximum inspiratory level and the maximum expiratory level. The third frame waveform F3 is constituted by a plurality of frame images in the maximum exhalation period. The fourth frame waveform F4 is constituted by a plurality of frame images at a substantially intermediate position between the maximum expiratory level and the maximum inspiratory level. Note that the number of frame waveforms to be set is not limited to that in FIG. 11. Furthermore, the setting position of the frame waveform is also not limited to FIG. 11, and it can be set at any arbitrary position (period) of the signal value change waveform.

The controller 31 compares frame waveforms in the same period set for each respiratory cycle. Based on the comparison result, the controller 31 determines, for each respiratory cycle, whether any of the plurality of frame waveforms in each respiratory cycle has an abnormality (step S201). To be specific, as shown in FIG. 11, the controller 31 compares the corresponding first frame waveforms F1, the corresponding second frame waveforms F2, the corresponding third frame waveforms F3, and the corresponding fourth frame waveforms F4, which are set for every n respiratory cycles. The value n is a positive integer. The controller 31 may determine the similarity between the frame waveforms on the basis of the number of peaks in each frame waveform, the width of the signal value of the waveform, or the like.

When the frame waveform having a low degree of similarity to the corresponding frame waveform of another respiratory cycle is not included in the first frame waveform F1 or the like of the first respiratory cycle C1, the controller 31 determines that the periodic noise is not included in the first respiratory cycle C1. The controller 31 performs the same process as the first respiratory cycle C1 for other respiratory cycles other than the first respiratory cycle C1. When the periodic noise is not included in all the respiratory cycles, the controller 31 determines that there is no abnormality in all the respiratory cycles. In this case, the controller 31 proceeds to step S28 in FIG. 7.

On the other hand, when the frame waveform having the low degree of similarity to the corresponding frame waveform of another respiratory cycle is included in the first frame waveform F1 or the like of the first respiratory cycle C1, the controller 31 determines that the periodic noise is included in the first respiratory cycle C1. The controller 31 performs the same process as the first respiratory cycle C1 for other respiratory cycles other than the first respiratory cycle C1. When the periodic noise is included in at least one or more respiratory cycles, the controller 31 determines that there is the abnormality in any of the respiratory cycles. In this case, the controller 31 extracts the respiratory cycle including the periodic noise, and proceeds to step S202.

The controller 31 generates non-analyzable information indicating that the extracted frame waveform of the respiratory cycle having the abnormality is inappropriate for the dynamic analysis processing (step S202). For example, the controller 31 may extract the respiratory cycle with little periodic noise by adding the non-analyzable information to all frame waveforms of the specified respiratory cycle. After generating the unanalyzable information, the controller 31 proceeds to step S28 in FIG. 7.

In the case of branching to the step S24, the controller 31 executes the third processing on the acquired dynamic image (step S27). Specifically, the controller 31 determines, as the standard frame image, the frame image of a normal portion assumed not to include the body motion in the signal value waveform in a case in which the region of interest R of each frame is set as the entire image. The controller 31 calculates the similarity to the standard frame image in each frame of the specific number of frames to be used for the dynamic analysis. The similarity may be determined using the signal value or the like, as described above. Based on the comparison result of the similarity, the controller 31 determines that the frame image whose similarity is low and exceeds the threshold value is an abnormal frame image including the noise due to body motion, and extracts the frame image whose similarity is low. The controller 31 may reject the extracted frame image, or may add non-analyzable information to the extracted frame image as described above.

The controller 31 performs dynamic analysis processing according to the purpose using the generated signal value change waveform (step S28). In a case where the first process and the second process are executed, the controller 31 executes the dynamic analysis processing on the frame image in which the non-analyzable information is not added to the dynamic image. That is, the controller 31 executes the dynamic analysis processing on the frame image not including the noise due to the patient's body motion or the like.

The controller 31 causes the generated signal value change waveform and the dynamic analysis result to be displayed on the screen of the display part 34 (step S29). For example, in a case where the first process and the second process are executed, the controller 31 displays the result of the dynamic analysis processing executed on the frame image to which the non-analyzable information is not added on the screen of the display part 34. At this time, the controller 31 may display, in a pop-up window on the screen of the display part 34, a message indicating that the frame image to which the non-analyzable information has been added is not being used for the dynamic analysis processing.

According to the second embodiment, the same effects as those of the first embodiment can be achieved. That is, at the timing before the dynamic analysis processing, the noise due to the patient's body motion or the like in each frame image of the dynamic image can be reduced. Accordingly, since the predetermined dynamic analysis can be performed using the appropriate dynamic image, the accurate result of the dynamic analysis can be obtained. Furthermore, according to the second embodiment, the frame image including the noise due to random or periodic body motion is further excluded using the signal value change waveform based on the region of interest in which the noise such as the body motion is reduced. Thus, since the noise such as the body motion that cannot be extracted by the tracking according to the first embodiment can be extracted, the dynamic image with high accuracy can be obtained.

Although the preferred embodiments of the present disclosure have been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

For example, although the dynamic analysis apparatus 3 performs the extraction processing of the noise such as the random body movement fluctuation of the patient included in the dynamic image in the above embodiment, it is not limited thereto. For example, the console 2 may function as the dynamic image processing apparatus and execute tracking of the region of interest of the standard frame image on another frame image. Furthermore, an information processing apparatus such as a client terminal may function as the dynamic image processing apparatus and perform tracking of the region of interest of the standard frame image on another frame image.

Although embodiments of the present disclosure have been described and illustrated in detail, the disclosed embodiments are made for purposes of illustration and example only and not limitation. The scope of the present disclosure should be interpreted by terms of the appended claims.

What is claimed is:

1. A dynamic image processing apparatus comprising:
a hardware processor that is configured to perform,
acquiring a dynamic image including a plurality of frame images obtained by dynamic imaging using radiation,
setting a certain region of interest in each of the plurality of frame images that are acquired, and
generating waveform information indicating a change in a signal value of each pixel in the set region of interest of the plurality of frame images,
wherein the hardware processor tracks the region of interest set in a standard frame image among the plurality of frame images with respect to another frame image to set the region of interest in the another frame image.

2. The dynamic image processing apparatus according to claim 1, wherein the region of interest is a local region within the frame image.

3. The dynamic image processing apparatus according to claim 2, wherein, in a case in which an imaging area of the dynamic image is a lung field region, the region of interest is a pulmonary artery.

4. The dynamic image processing apparatus according to claim 1, wherein the hardware processor extracts a frame image including a dose fluctuation different from a blood flow fluctuation and a respiration-induced fluctuation included in the generated waveform information.

5. A dynamic image processing method comprising:
acquiring a dynamic image including a plurality of frame images obtained by dynamic imaging using radiation,
setting a certain region of interest in each of the plurality of frame images that are acquired, and
generating waveform information indicating a change in a signal value of each pixel in the set region of interest of the plurality of frame images,
wherein in the setting, the region of interest set in a standard frame image among the plurality of frame images is tracked with respect to another frame image to set the region of interest in the another frame image.

6. A non-transitory computer readable storage medium including a program that controls a computer to perform,
acquiring a dynamic image including a plurality of frame images obtained by dynamic imaging using radiation,
setting a certain region of interest in each of the plurality of frame images that are acquired, and
generating waveform information indicating a change in a signal value of each pixel in the set region of interest of the plurality of frame images,
wherein in the setting, the region of interest set in a standard frame image among the plurality of frame images is tracked with respect to another frame image to set the region of interest in the another frame image.
